(19) United States
(12) Patent Application Publication
(10) Pub. No.: US 2010/0087501 A1
(43) Pub. Date: Apr. 8, 2010

(54) Highly Bioavailable Composition Containing Eprosartan-Poloxamer Complex or 2-(7-Chloro-5-Methyl-4-Oxo-3-Phenyl-4,5-Dihydro-3H-Pyridazine (4,5-b)Indol-1-yl)-N,N-Dimethylacetamide - Poloxamer Complex

(75) Inventors: Ketan Mehta, Cranbury, NJ (US); Yu Hsing Tu, West Windsonr, NJ (US)

Correspondence Address: HOWSON & HOWSON LLP, 501 OFFICE CENTER DRIVE, SUITE 210, FORT WASHINGTON, PA, 19034, US

(73) Assignee: BVM Holding Co., Monmouth Junction, NJ (US)

(21) Appl. No.: 12/633,116

(22) Filed: Dec. 8, 2009

Related U.S. Application Data
(63) Continuation of application No. 10/961,977, filed on Oct. 9, 2004, now abandoned.
(60) Provisional application No. 60/510,407, filed on Oct. 10, 2003.
(60) Provisional application No. 60/510,408, filed on Oct. 10, 2003.
(60) Provisional application No. 60/525,351, filed on Nov. 26, 2003.

(51) Int. Cl. A61K 31/4178; A61P 9/12

(52) U.S. Cl. 514/397

(57) ABSTRACT

A composition including an association complex of a pharmaceutical composition and one or more polyethylene-polypropylene glycol block copolymers (poloxamers) is provided. The pharmaceutical composition may include a member selected from the group consisting of (a) an association complex of an eprosartan composition including eprosartan or a pharmaceutically acceptable salt of eprosartan and (b) the non-zwitterionic compound 2-(7-chloro-5-methyl-4-oxo-3-phenyl-4,S dihydro-3H-pyridazino(4,S-b)indol-1-yl)N,N-dimethylacetamide (NZ).

### CROSS REFERENCE TO RELATED APPLICATIONS

This application is a continuation of U.S. patent application Ser. No. 10/961,977, filed Oct. 9, 2004, which claims the benefit of to U.S. Provisional Patent Application Nos. 60/510,407 filed on Oct. 10, 2003; 60/510,408 filed on Oct. 10, 2003 and 60/525,351 filed on Nov. 26, 2003, which are herein incorporated by reference in there entirety.

### FIELD OF THE INVENTION

This invention relates to pharmaceutical compositions; more specifically, to improving the dissolution and dissolution rate of water insoluble drugs using complexation as an approach to achieving that goal.

### BACKGROUND OF THE INVENTION

Good absorption and adequate blood levels are essential for any drug to have its intended pharmacological effect. Before being absorbed into the body through the gastrointestinal (GI) tract drugs need to be dissolved in the aqueous fluids of the GI tract. The amount of dissolution and, often, the rate of dissolution of a drug in aqueous media, are critical to its absorption and the resulting blood levels of that drug. Newer drugs are increasingly hydrophobic and/or insoluble possessing poor or almost no dissolution in water or the aqueous fluids of the GI tract.

While many approaches are used for solubilizing or improving the dissolution of such drugs, one approach involves the use of surfactants such as polyethylene-polypropylene glycol block co-polymers, commonly known as poloxamers, as a solubilizer or stabilizing agent. This has been subject of publications such as J. Pharma. Sci., 1976, 65, 115:118. U.S. Pat. No. 6,255,284 describes the use of poloxamers, in varying low concentrations of less than 1% as a stabilizing agent in resisting peptide and polypeptide aggregation in aqueous solutions. U.S. Pat. No. 6,432,381 describes the use of poloxamers at levels below 10%, and preferably below 5%, as a stabilizer and/or as a bioadhesive material.

The use of complexation as an approach to increase the dissolution has generally included the use of cyclodextrin and its various derivatives. The complexes formed using cyclodextrin are inclusion complexes wherein the drug is more enclosed in the ring or cavity of the complexing or binding agent to affect solubilization as described in U.S. Pat. No. 6,407,079. This approach works in limited situations and does not provide the high levels of drug loading that are required with many therapeutic agents.

One drug having low aqueous solubility is a zwitterionic compound, eprosartan mesylate, (EPM), empirical formula C₂₃H₂₄N₂O₄S.CH₄O₃S. EPM is a non-biphenyl nontetrazole angiotensin II receptor (AT) antagonist and has powerful anti-hypertensive effect if present in sufficient amount in the blood. However, it has extremely poor water solubility, having an aqueous solubility below 1 mg/L or below 1 μg/mL water. This insolubility has resulted in poor dissolution and absorption of the drug in the body and consequently approximately only 10% of the drug is bioavailable when given orally. Techniques such as micronization or the use of surfactants have not achieved any significant improvement in the solubility and dissolution of these drugs.

Another drug with poor solubility is the non-zwitterionic compound 2-(7-chloro-5-methyl-4-oxo-3-phenyl-4,5 dihydro-3H-pyridazino (4,5-b)-indol-1-yl)-N,N-dimethylacetamide, which will be referred to as “NZ” hereinafter. NZ is also extremely poorly soluble, having aqueous solubility below 1 mg/L or below 1 μg/mL water.

Since oral administration is preferred for the administration of medications, there is a need for compositions including EPM, NZ, and other poorly soluble pharmaceutical compositions which provide adequate dissolution and bioavailability in an oral dosage form.

### SUMMARY OF THE INVENTION

It has now been found that the use of association complexes using the solid form of poloxamers at levels in the weight ratio of drug to poloxamers of 10:1 to 1:15 significantly improves the dissolution and dissolution rates of pharmaceutical compounds such as EPM and NZ. The weight ratio of drug to poloxamers is about preferably 10:3 to about 1:9; more preferably about 10:3 to about 1:3; even more preferably about 1:1.

One aspect of the present invention provides a composition including an association complex of a pharmaceutical composition which may be the non-zwitterionic compound 2-(7-chloro-5-methyl-4-oxo-3-phenyl-4,5-dihydro-3H-pyridazino(4,5-b) indol-1-yl)N,N-dimethylacetamide (NZ) or an eprosartan composition including eprosartan or a pharmaceutically acceptable salt of eprosartan and one or more solid form of polyethylenepolypropylene glycol block co-polymers (poloxamers).

Another aspect of the present invention provides a method of improving the dissolution of pharmaceutical compositions such as an eprosartan composition including eprosartan or pharmaceutically acceptable salt of eprosartan or NZ. The method includes adding the pharmaceutical composition to one or more poloxamers to provide an association complex.

A further aspect of the present invention include a method of improving the bioavailability of eprosartan or a pharmaceutically acceptable salt of eprosartan which includes adding eprosartan or pharmaceutically acceptable salt of eprosartan or NZ to one or more solid form of poloxamers to provide an association complex. A pharmaceutically acceptable salt of eprosartan is eprosartan mesylate.

A still further aspect of the present invention provides a pharmaceutical composition including: (a) an association complex of the non-zwitterionic compound 2-(7-chloro-5-methyl-4-oxo-3-phenyl-4,5 dihydro-3H-pyridazino(4,5-b)indol-1-yl)-N,N-dimethylacetamide (NZ) or an eprosartan composition including eprosartan or a pharmaceutically acceptable salt of eprosartan and one or more solid form of polyethylenepolypropylene glycol block co-polymers (poloxamers); and (b) a pharmaceutically acceptable carrier. The association complex may have a weight ratio of eprosartan composition to poloxamer of about 10:1 to about 1:15.

Also provided is a method of treating hypertension including orally administering an effective amount of a composition including: (a) an association complex of an eprosartan composition including eprosartan or a pharmaceutically acceptable salt of eprosartan and one or more solid form of polyethylene-polypropylene glycol block co-polymers (poloxamers); and (b) a pharmaceutically acceptable carrier.

Another aspect of the present invention provides a method of preparing an association complex including a pharmaceutical composition including:

(a) providing a pharmaceutical composition; and

(b) heating and mixing said pharmaceutical composition with one or more solid form of polyethylene-polypropylene glycol block co-polymers (poloxamers) to provide an association complex.

### DETAILED DESCRIPTION OF INVENTION

For the purposes of the present invention, the term “association complex” or “complex” is meant to include a combination of compounds which are bound together more strongly than would be expected to result from mere physical mixing. Without being limited to a specific theory, the binding forces may be described as “weak forces”, e.g., van der Waals forces or hydrogen bonding.

For the purposes of the present invention, the term “poorly soluble” as used to describe pharmaceutical compositions, i.e., drugs, includes compositions having a solubility in aqueous medium of less than about 10 mg/ml, preferably of less than about 1 mg/ml, and more preferably less than 0.1 mg/ml.

The present invention provides formation of an association complex between hydrophobic or poorly soluble drugs such as eprosartan mesylate (BPM), a zwitterionic compound having multiple pKa, mol. wt. 520.6 and a high melting point or such as the nonzwitterionic compound 2-(7-chloro-5-methyl-4-oxo-3-phenyl-4,5 dihydro-3H-pyridazino(4,5b)indol-1-yl)-N,N-dimethylacetamide (NZ), and solid form of polyoxyethylene-polyoxypropylene co-polymers (poloxamers). The poorly soluble pharmaceutical compositions or drugs of the present invention will desirably have high melting points such as above 70° C. and more preferably above 100° C. The poloxamers are used at high levels in combination with the poorly soluble pharmaceutical composition. The association complex thus formed exhibits superior dissolution as compared to the drug itself or to conventional drug/excipient mixtures. The complex thus produced may be further mixed with suitable commonly used pharmaceutically used excipients such as disintegrants, binders, diluents and lubricants as further provided in U.S. Pat. No. 6,274,168B1, herein incorporated by reference in its entirety. The association complex can be filled into a capsule, compressed into a tablet, formulated into any other suitable oral dosage form.

The poorly soluble pharmaceutical composition may be any of a wide variety of drugs. these include analgesics, anti-inflammatory agents, anthelmintics, anti-arrhythmic agents, antibiotics (e.g., penicillins), anticoagulants, antidepressants, antidiabetic agents, antiepileptics, antifungal agents, antihistamines, antihypertensive agents, antiinfectives, antimuscarinic agents, antimycobacterial agents, antineoplastic agents, antiviral agents, immunosuppressants, antithyroid agents, anxiolytic sedatives (hypnotics and corticosteroids, cough suppressants (expectorants and mucolytics), dopaminergics (antiparkinsonian agents), haemostatics, immunological agents, lipid regulating agents, muscle relaxants, parasympathomimetics, parathyroid calcitonin and biphosphonates, protease inhibitors, prostaglandins, radio-pharmaceuticals, sex hormones (including steroids), anti-allergic agents, stimulants and anorectics, sympathomimetics, thyroid agents, vasodilators, and xanthines.

A variety of solid forms of poloxamers are useful in the present invention. These include poloxamers having an average molecular weight of from about 1,000 to 15,000 Daltons, desirably from about 5,000 to 15,000 Daltons. Poloxamers are polyethylene-polypropylene glycol block co-polymers containing ethylene oxide (PEO) and propylene oxide (PPO) segments according to the formula

(PEO)ₐ-(PPO)_{b}-(PEO)_{c}

Preferred poloxamers are those wherein:

a and c are about 45 to about 130; and b is about 15 to 70.

Given the nature of a block co-polymer, the a and c segments are going to be approximately the same. Examples of suitable poloxamers include those with a and c from about 75 to about 80 and b from about 25-30 as well as poloxamers with a and c from about 98-101 and b from about 56 to about 67.

Commercially available poloxamers for use in the present invention include Lutrol (BASF Corporation): Lutrol® F127, F108, F98, F87, F88, F77, F68, and F38. Desirably, the poloxamers include Poloxamer 188 (Lutrol F68) and Poloxamer 407 (Lutrol® F127).

Characteristics of suitable poloxamer surfactants include those characterized by a HLB value greater than about 14 and a surface tension between about 10 and about 70 mN/m as measured in aqueous solution at room temperature and at a concentration of 0.1%. Desirably, the HLB value is between about 25 and 35 and the poloxamer has surface tension between 30 and 52 mN/m as measured in aqueous solution at room temperature and at a concentration of 0.1%. An example of a desirable poloxamer is poloxamer 188 (USP/NF) available commercially as Lutrol F-68, which has a surface tension of 50 mN/m and by an HLB value of 29.

The present invention provides an association complex of a drug or pharmaceutical composition such as EPM or NZ and one or more poloxamers, which improves the solubility and dissolution rate of the drug. For the formation of the association complex, the compounds are mixed in a suitable mixer such as the jacketed high shear granulator of Key International's Model #KG5 (KG5) and heated to about 50-65° C. until free flowing granulation is formed. By free flowing granulation, it is meant that the granules formed have an angle of repose in the range of about 30° to about 45°; and particle size ranges from about 100 microns to about 1000 microns for at least 50%, 25% above 1000 microns and 25% below 100 microns. The temperature between about 50-65° C. is selected to ensure melting of the polymer while maintaining the solid crystalline form of the drug that is being complexed with the polymer. The resultant complex is cooled to room temperature and sieved to a suitable particle size, such as through #20 or #40 mesh size screens, preferably in the range of #20 to #60.

The dissolution rate of the association complex may be carried out using a suitable solvent such as O.1 N HCl purified water, or simulated gastric fluid, as dissolution medium. The testing is conducted under conditions intended to simulate ingestions at about 37° C. and from about 25 to about 75 rpm. The dissolution may be tested using any method known in the art such as USP apparatus I (a basket apparatus) or USP apparatus II (a paddle apparatus) (USP 27/NF 22, 2004, <711>, page 2303). Samples of the solution are taken at preselected intervals such as at every fifteen minutes up to one hour.

As described above, the association complex including EPM and one or more poloxamers can be formulated into any suitable oral dosage form such as tablets and capsules. The oral dosage form is a pharmaceutical composition useful in the treatment of hypertension. Generally, EPM is administered in amounts from about 400 mg to 600 mg either once or twice per day. As the bioavailability of the compound of EPM is only about 10%, an increase of bioavailability of an additional 10% would decrease the necessary dosage by half, a significant decrease in the amount of drug needed to be taken to achieve the same desired pharmacological effect.

The dissolution and bioavailability of pharmaceutical compositions such as EPM or NZ are improved by the complexing of the pharmaceutical composition with one or more poloxamers. Within the association complex, the wt/wt ratio of the pharmaceutical composition to poloxamer ranges from 10:1 to 1:15; preferably 10:3 to 1:9; more preferably 10:3 to 1:3; most preferably 1:1.

Wherein the pharmaceutical composition includes eprosartan, the present invention is not limited to complexes of the mesylate salt of eprosartan. The complexes of the invention may also be formed with eprosartan and also with its other pharmaceutically acceptable salts. Eprosartan is (E)-α-[[2-butyl-1-[(4-carboxyphenyl)methyl]-1H-imidazol-5-yl]methylene]-2-thiophene propanoic acid. The compound is described in U.S. Pat. No. 5,185,351, herein incorporated by reference in its entirety. Thus, the invention provides formation of an association complex between eprosartan or any of its pharmaceutically acceptable salts and polyoxyethylene-polyoxypropylene co-polymers (poloxamers) when the poloxamers are used at relatively high levels as set forth in the weight ratios described above.

The acid addition salts of eprosartan are formed with the appropriate inorganic or organic acids by methods known in the art. Representative examples of suitable acids are maleic, fumaric, acetic, succinic, hydrochloric, hydrobromic, sulfuric, phosphoric, and methanesulfonic. The pharmaceutically acceptable acid addition salt for eprosartan mesylate is the methanesulfonic acid addition salt.

The base addition salts of eprosartan are formed with the appropriate inorganic or organic bases by methods known in the art. Cationic salts are prepared, for example, by treatment with an excess of an alkaline reagent, such as hydroxide, carbonate, or alkoxide, containing the appropriate cation; or with an appropriate organic amine. Representative examples of cations are Li⁺, Na⁺, K⁺, Ca⁺⁺, Mg⁺⁺, and NH₄⁺.

The features and advantages of the present invention are more fully shown by the following examples which are provided for purposes of illustration, and are not to be construed as limiting the invention in any way.

### Example 1

### Eprosartan Mesylate (1:1) Complex

Lutrol F-68, 1.0 gm, and EPM, 1.0 gm, are mixed in a jacketed vessel (KG5) heated to 53° C. while mixing for about 2 hrs. The resulting complex, in the form of free flowing granules, thus formed is allowed cool to room temperature and sieved through a 40 mesh screen. The dissolution of the complex equivalent to the 400 mg dose is determined using USP apparatus II, 900 ml of SGF (simulated gastric fluid without pepsin) as the dissolution medium at 37° C. and 25 rpm. Table I provides the dissolution rate of EPM.

TABLE 1
EPM (1:1) Complex Dissolution Rate

| TIME (minutes) | % DISSOLVED |
|---|---|
| 15 | 85.95 |
| 30 | 95.26 |
| 45 | 95.80 |
| 60 | 97.05 |

### Example 2

### Eprosartan Mesylate (3:7) Complex

Lutrol F-68, 1.4 gm, and EPM, 600 mg, are mixed in a jacketed vessel (KG 5) heated to 53° C. while mixing for about 2 hrs. The resulting complex thus formed is cooled to room temperature and sieved through a 40 mesh screen.

The dissolution of the complex equivalent to a 400 mg dose is determined using USP apparatus II, in SGF as the dissolution medium at 37° C. and 25 rpm. Table 2 provides the dissolution rate of EPM.

TABLE 2
EPM (3:7) Complex Dissolution Rate

| TIME (minutes) | % DISSOLVED |
|---|---|
| 15 | 92.5 |
| 30 | 93.9 |
| 45 | 94.4 |
| 60 | 94.6 |

### Comparative Example 3

Table 3 provides a comparison of dissolution rate of the compound EPM by itself and that of association complex formed per Examples 1 and 2. An increase of more than 70% is obtained. Further, the dissolution rate was substantially faster for the complex than for the compound itself. This increase in the rate of dissolution is often very critical for drugs such as EPM where a narrow window of absorption has been implicated for poor absorption. By having a significantly faster rate of the drug dissolution at or around the site of absorption, the complex helps provide a higher concentration of the drug than for non-complexed compounds; thus providing higher absorption or blood levels.

TABLE 3
Complex vs. Compound EPM Dissolution Rates

| | % DISSOLVED | | |
|---|---|---|---|
| TIME (minutes) | EPM | 1:1 COMPLEX | 3:7 COMPLEX |
| 15 | 38.7 | 86.0 | 92.5 |
| 30 | 51.9 | 95.3 | 93.9 |
| 45 | 61.3 | 95.8 | 94.4 |
| 60 | 61.7 | 97.1 | 94.6 |

### Example 4

Lutrol F-68 and NZ, 250 mg are mixed in a jacketed vessel (KG 5) and heated to 53° C. while mixing for about 2 hours. The resulting complex thus formed is allowed to cool to room temperature and sieved through a 40 mesh screen. The dissolution of the complex equivalent to 20 mg drug is determined using USP apparatus II, 1000 ml of purified water as the dissolution medium at 37° C. and 75 rpm. Table 4 provides the dissolution rate of NZ.

TABLE 4
NZ (1:9) Complex Dissolution Rate

| TIME (minutes) | % DISSOLVED |
|---|---|
| 15 | 1.28 |
| 30 | 7.53 |
| 45 | 8.96 |
| 60 | 11.01 |

Table 5 provides a comparison of dissolution rate of the compound NZ by itself and that of the association complex as formed in Example 4. An increase of almost two-fold or more is obtained.

TABLE 5
Complex vs. Compound NZ Dissolution Rates

% DISSOLVED

| TIME (minutes) | NZ | 1:9 COMPLEX |
|---|---|---|
| 15 | 0.65 | 1.28 |
| 30 | 1.90 | 7.53 |
| 45 | 2.53 | 8.96 |
| 60 | 3.14 | 11.01 |

While there have been described what are presently believed to be the preferred embodiments of the invention, those skilled in the art will realize that changes and modifications may be made thereto without departing from the spirit of the invention, and it is intended to include all such changes and modifications as fall within the true scope of the invention.

What is claimed is:

1. A method of preparing an association complex of an eprosartan composition comprising eprosartan or a pharmaceutically acceptable salt of eprosartan and one or more polyethylene-polypropylene glycol block co-polymers (poloxamers) comprising:
mixing the eprosartan or the salt of eprosartan and one or more polyethylene-polypropylene glycol block co-polymers (poloxamers) in a weight ratio of said eprosartan composition to said poloxamer of about 10:1 to about 1:9 and heating to form the association complex.

2. The method according to claim 1, wherein the heating is to a temperature of about 50° C. to 65° C.

3. The method according to claim 1, wherein the complex forms a free flowing granulation.

4. The method of claim 1, wherein the weight ratio of eprosartan mesylate to poloxamer is about 3:7.

5. The method of claim 1, wherein said complex has a weight ratio of eprosartan mesylate to poloxamer is about 1:9.

6. A pharmaceutical composition which is an oral dosage form and which comprises (a) an association complex of an eprosartan composition comprising eprosartan or a pharmaceutically acceptable salt of eprosartan and one or more solid form of polyethylene-polypropylene glycol block co-polymers (poloxamers) wherein said association complex has a weight ratio of eprosartan composition to said poloxamer of about 10:3 to about 1:9; and (b) a pharmaceutically acceptable carrier.

7. The pharmaceutical composition of claim 6, wherein said eprosartan composition comprises eprosartan mesylate.

8. The pharmaceutical composition of claim 6, wherein the weight ratio of eprosartan mesylate to poloxamer is about 3:7.

9. The pharmaceutical composition of claim 6, wherein said complex has a weight ratio of eprosartan mesylate to poloxamer is about 1:9.

10. The pharmaceutical composition of claim 6, wherein said complex is a free flowing granulation.

12. The pharmaceutical composition according to claim 6, wherein the poloxamers have an average molecular weight of from about 1000 to 15,000 Daltons.

13. The pharmaceutical composition according to claim 11, wherein the poloxamers have an average molecular weight of from about 5000 to 15,000 Daltons.

14. The pharmaceutical composition according to claim 6, wherein the poloxamers are characterized by the repeating formula:
(ethylene oxide)ₐ-(propyleneoxide)_{b}-(ethyleneoxide)_{c}, wherein a and c are about 45 to 130 and b is about 15 to 70.

14. The pharmaceutical composition according to claim 13, wherein a and c are about 75 to about 80 and b is about 25 to 30.

15. The pharmaceutical composition according to claim 13, wherein a and c are about 98 to about 101 and b is about 56 to 67.

16. The pharmaceutical composition according to claim 6, wherein the granules of the free flowing granulation have a particle size ranging from about 100 microns to about 1000 microns for at least 50%, 25% above about 1000 microns and 25% below about 100 microns.

17. A method of preparing an association complex of the non-zwitterionic compound 2-(7-chloro-5-methyl-4-oxo-3-phenyl-4,5 dihydro-3H-pyridazino(4,5-b)indol-1-yl)-N,N-dimethylacetamide (NZ) and one or more polyethylene-polypropylene glycol block co-polymers (poloxamers) comprising:
mixing the non-zwitterionic compound 2-(7-chloro-5-methyl-4-oxo-3-phenyl-4,5 dihydro-3H-pyridazino(4,5-b)indol-1-yl)-N,N-dimethylacetamide (NZ) and one or more polyethylene-polypropylene glycol block co-polymers (poloxamers) in a weight ratio of said NZ to said poloxamer of about 10:1 to about 1:9 and heating to form the association complex.

18. The method according to claim 17, wherein the heating is to a temperature of about 50° C. to 65° C.

19. The method according to claim 17, wherein complex forms a free flowing granulation.

20. A method of treating hypertension comprising orally administering an effective amount of a composition according to claim 6.
